(19) United States
(12) Patent Application Publication
(10) Pub. No.: US 2019/0125218 A1
(43) Pub. Date: May 2, 2019

(54) CRAMP EVALUATING DEVICE FOR CALF MUSCLE, EVALUATING SYSTEM AND EVALUATING METHOD USING THE SAME

(71) Applicant: INSTITUTE FOR INFORMATION INDUSTRY, Taipei (TW)

(72) Inventors: Yu-Cheng TSAI, Xingang Township (TW); Ping-Che YANG, Taipei City (TW)

(73) Assignee: INSTITUTE FOR INFORMATION INDUSTRY, Taipei (TW)

(21) Appl. No.: 15/829,261

(22) Filed: Dec. 1, 2017

(30) Foreign Application Priority Data
Nov. 1, 2017 (TW) 106137828

(51) Int. Cl. A61B 5/11; A61B 5/103; A61B 5/0488

(52) U.S. Cl. CPC A61B 5/1107; A61B 5/6802; A61B 5/0488; A61B 5/1038

(57) ABSTRACT

A cramp evaluating device for calf muscle, adapted to evaluate a probability value of calf muscle of a runner wearing a number of sensors, includes a physiological signal receiver and a cramp detector. The physiological signal receiver is configured to receive a plurality of physiological signals from the sensors. The cramp detector is configured to input the physiological signals to an evaluation model to calculate the probability value of the calf muscle of the runner.

This application claims the benefit of Taiwan application Serial No. 106137828, filed Nov. 1, 2017, the disclosure of which is incorporated by reference herein in its entirety.

### TECHNICAL FIELD

The disclosure relates in general to a cramp evaluating device, an evaluating system and an evaluating method using the same, and more particularly to a cramp evaluating device for calf muscle, an evaluating system and an evaluating method using the same.

### BACKGROUND

Under the guidance of the concept of health, road running has become one of the common modern sports. Running sometimes results in a cramp to runner's leg. However, the runner feels the cramp after the cramp occurs. Therefore, it is necessary to propose a new technique for evaluating the probability of a runner's calf muscle cramp before the cramp occurs, so as to warn the runner to avoid the occurrence of cramp before the calf muscle cramp.

### SUMMARY

The disclosure is directed to a cramp evaluating device for calf muscle, an evaluating system and an evaluating method using the same to solve the above problem.

According to one embodiment, a cramp evaluating device for calf muscle is provided. The cramp evaluating device for calf muscle, adapted to evaluate a probability value of calf muscle of a runner wearing a number of sensors, includes a physiological signal receiver and a cramp detector. The physiological signal receiver is configured to receive a plurality of physiological signals from the sensors. The cramp detector is configured to input the physiological signals to an evaluation model to calculate the probability value of the calf muscle of the runner.

According to another embodiment, a cramp evaluating system for calf muscle is provided. The cramp evaluating device, adapted to evaluate a probability value of calf muscle of a runner wearing a number of sensors, includes a physiological signal receiver and a device wireless signal transceiver. The physiological signal receiver is configured to receive a plurality of physiological signals from the sensors. The device wireless signal transceiver is configured to transmit the physiological signals to a cramp evaluating system for calf muscle, wherein the cramp evaluating system inputs the physiological signals to an evaluation model for calculating the probability value of the calf muscle of the runner, and the device wireless signal transceiver is configured to receive the probability value.

According to another embodiment, a cramp evaluating system for calf muscle is provided. The cramp evaluating system, adapted to evaluate a probability value of calf muscle of a runner wearing a number of sensors, includes a system wireless signal transceiver and a cramp detector. The system wireless signal transceiver is configured to receive a plurality of physiological signals from the sensors. The cramp detector is configured to input the physiological signals to an evaluation model to calculate the probability value of the calf muscle of the runner. The system wireless signal transceiver is further configured to transmit the probability value to a cramp evaluating device.

According to another embodiment, a cramp evaluating method for calf muscle is provided. The cramp evaluating method, adapted to evaluate a probability value of calf muscle of a runner wearing a number of sensors, includes the following steps. A plurality of physiological signals from the sensors is received; and the physiological signals are inputted to an evaluation model to calculate the probability value of the calf muscle of the runner.

### BRIEF DESCRIPTION OF THE DRAWINGS

FIG. 1 A shows a block diagram of a cramp evaluating device for calf muscle according to an embodiment of the present invention;

FIG. 1B shows a block diagram of another cramp evaluating device for calf muscle according to an embodiment of the present invention;

FIG. 2 shows a block diagram of a cramp evaluating device and a cramp evaluating system for calf muscle according to another embodiment of the present invention;

FIG. 3 shows a flowchart of a calf muscle cramp evaluating method of the cramp evaluating device of FIG. 1 B;

FIG. 4 shows a flowchart of a cramp evaluating method of the cramp evaluating device for calf muscle of FIG. 2; and

FIG. 5 shows a diagram of an establishing process of the evaluating model according to an embodiment of the invention.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

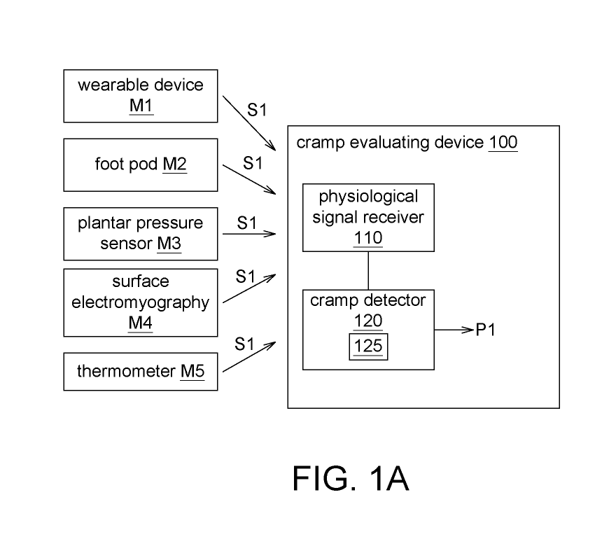

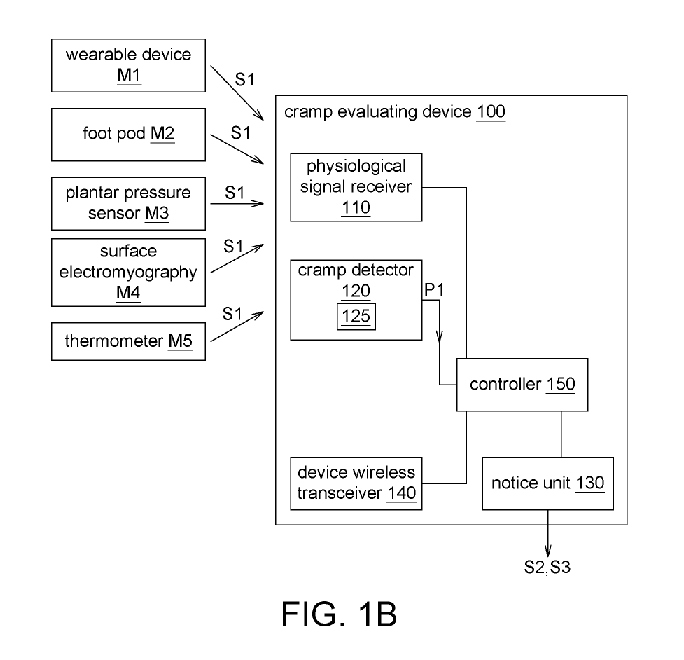

FIG. 1A shows a block diagram of a cramp evaluating device 100 for calf muscle according to an embodiment of the present invention. The cramp evaluating device 100 may evaluate a probability of cramp to calf muscle of a runner. The runner may wear a number of sensors, such as a wearable device M1, a foot pod M2, a plantar pressure sensor M3, a surface electromyography (SEMG) M4, a thermometer M5 or other kinds of sensors or elements. The wearable device M1 is, for example, a smart watch, a smart bracelet, a smart phone, or other portable electronic device.

The cramp evaluating device 100 includes a physiological signal receiver 110 and a cramp detector 120, wherein the cramp detector 120 includes an evaluating model 125.

The physiological signal receiver 110 is configured to receive a plurality of physiological signals S1 from the sensors (M1-M5). The cramp detector 120 is configured to input the physiological signals S1 to the evaluating model 125 for calculating the probability P1 of cramp to calf muscle of the runner. In addition, the evaluating model 125 may be stored in a storing element of the cramp detector 120, such as a memory, or a controller 150 (as shown in FIG. 1B) or another storing element disposed on the cramp evaluating device 100.

FIG. 1B shows a block diagram of the cramp evaluating device 100 according to another embodiment of the present invention. The cramp evaluating device 100 includes the physiological signal receiver 110, the cramp detector 120, a notice unit 130, a device wireless transceiver 140 and the controller 150. The physiological signal receiver 110 and the cramp detector 120 have functions as described above. The controller 150 may control, process, transmit and/or analyze the signals of the physiological signal receiver 110, the cramp detector 120, the notice unit 130 and the device wireless transceiver 140. When the probability P1 calculated by the cramp detector 120 is larger than a threshold, the controller 150 controls the notice unit 130 to output a warning signal S2 for warning the runner of cramp being possible to occur, and accordingly it can avoid sports injuries. The probability P1 is, for example, equal to or larger than 71%. In another embodiment, when the probability P1 is less than the threshold, the controller 150 controls the notice unit 130 to output a safe signal S3, or the notice unit 130 may not output the safe signal S3. As described above, the notice unit 130 may output a notice signal, such as the warning signal S2 or the safe signal S3 according to the probability P1.

In addition, the controller 150 may be a central processing unit (CPU) provided in a general computer apparatus/computer. The controller 150 may be programmed to interpret computer instructions, process data for computer software, and execute various arithmetic programs. The controller 150 may be a processor including a plurality of individual units or a microprocessor comprised of one or more integrated circuits.

The notice unit 130 is, for example, a display, a speaker, an earphone, a vibrator or a device which can output the warning signal S2. The above notice signal (such as the warning signal S2 and the safe signal S3) is, for example, an image, a voice, a vibration or a signal which can generate a notice function. In the example of the notice unit 130 being a display, when the probability P1 is larger than 81%, the notice unit 130 outputs, for example, a red light which means warning. When the probability P1 ranges between about 71% and about 81%, the notice unit 130 outputs, for example, a yellowish red light which means warning. When the probability P1 ranges between about 61% and about 71%, the notice unit 130 outputs, for example, a green light which means safe. When the probability P1 ranges between about 51% and about 61%, the notice unit 130 outputs, for example, a blue light with yellow tone which means safe. When the probability P1 is less than 50%, the notice unit 130 outputs, for example, a blue light which means safe.

In addition, the device wireless transceiver 140 may transmit the physiological signals S1 and/or the probability P1 to an exterior electronic device for a suitable using. In another embodiment, the cramp evaluating device 100 may omit the device wireless transceiver 140. In addition, the physiological signal receiver 110 may be wireless receiver using a communication technology, such as Bluetooth, Wi-Fi or ANT+ (according to ANT communication protocol developed by the ultra-low power short-range wireless transmission standard) for wirelessly receiving the physiological signals S1. In an embodiment, the physiological signal receiver 110 and the device wireless transceiver 140 may be integrated into single component.

In addition, types of the physiological signals S1 may include at least two of mean power frequency (MPF), maximal volitional contraction (MVC), heart rate variability (HRV), root mean square (RMS), medium frequency (MF), body temperature, foot pressure, Integrate EMG (iEMG), critical velocity (CV) and AT/AeT.

In these signals, the maximal volitional contraction, the mean power frequency, the root mean square and the medium frequency may come from EMG signal measured by the surface electromyography M4. The heart rate variability may come from heart rate signal measured by the wearable device M1 or the surface electromyography M4. The body temperature may come from the thermometer M5. The foot pressure may come from the plantar pressure sensor M3. The kinds of physiological signal receiver 110 and the sensors are not limited to the embodiments, the physiological signals S1 and other physiological signals not described may come from other kinds of sensors.

In addition, not all of the physiological signals S1 are involved in the operation of the probability P1. In one embodiment, depending on the requirements of the evaluating model 125, some of the physiological signals S1 may be selected to participate in the operation of the probability P1. Based on this, the runners can only wear the source sensors whose physiological signals S1 participate in the probability P1. If necessary, all of the physiological signal S1 may participate in the operation of the probability P1.

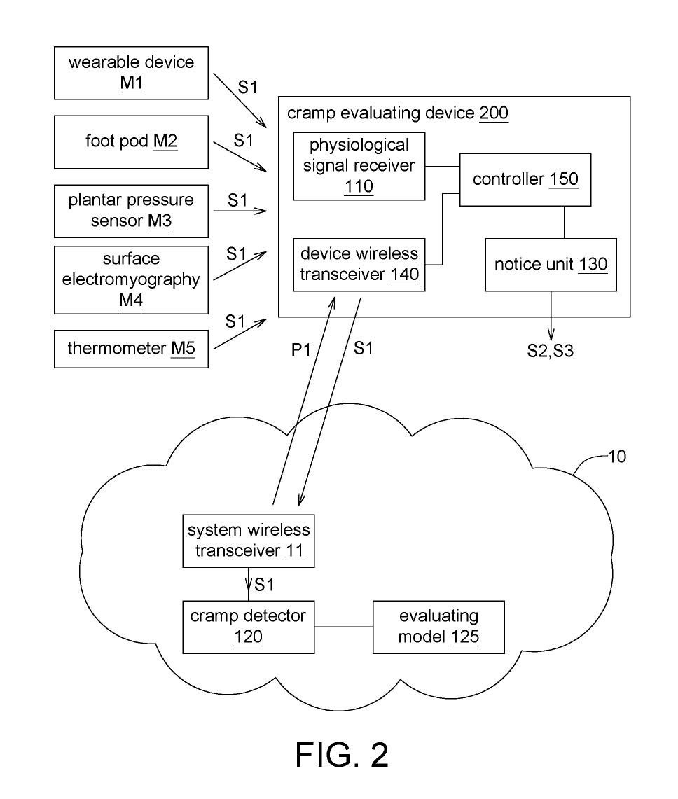

FIG. 2 shows a block diagram of a cramp evaluating device 200 and a cramp evaluating system 10 for calf muscle according to another embodiment of the present invention. It is different from the cramp evaluating device 100 in that the probability value P1 of the present embodiment is calculated by the cramp evaluating system 10. The cramp evaluating system 10 may be built in a cloud, or built on a typical personal computer, laptop or other electronic device having operating function.

The cramp evaluating device 200 includes the physiological signal receiver 110 and the device wireless transceiver 140. In another embodiment, the cramp evaluating device 200 further includes the notice unit 130 and the controller 150. The physiological signal receiver 110 is configured to receive a plurality of the physiological signals S1 from the sensors (M1-M5). The physiological signals S1 are transmitted to the cramp evaluating system 10 through the device wireless transceiver 140. The cramp evaluating system 10 includes a system wireless transceiver 11 and the cramp detector 120. The system wireless transceiver 11 is configured to receive the physiological signals S1 transmitted from the cramp evaluating device 200. The cramp detector 120 may input the physiological signals S1 to the evaluating model 125 for calculating the probability P1 of cramp to calf muscle of the runner. In addition, the evaluating model 125 may be stored in a storing element of the cramp detector 120, such as a memory, or be stored in the storing device of the cramp evaluating system 10 or other storing element (not shown). The probability P1 may be transmitted to the cramp evaluating device 200 through the system wireless transceiver 11. The device wireless transceiver 140 of the cramp evaluating device 200 may receive the probability P1. When the probability P1 is larger than the threshold, the controller 150 controls the notice unit 130 to output the warning signal S2 for warn the runner of cramp being possible to occur.

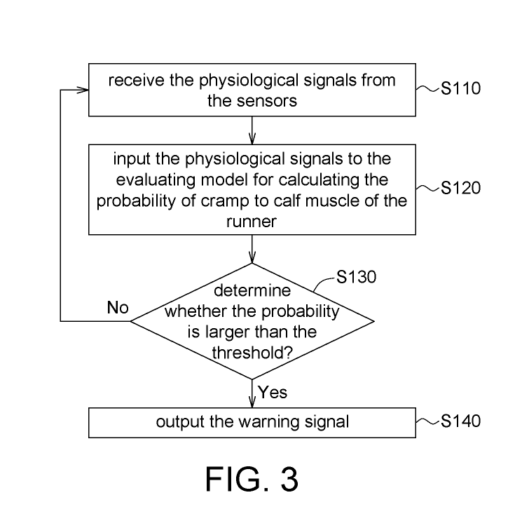

Referring to FIGS. 1B and 3, FIG. 3 shows a flowchart of a calf muscle cramp evaluating method of the cramp evaluating device 100 of FIG. 1B.

In step S110, the physiological signal receiver 110 of the cramp evaluating device 100 receives the physiological signals S1 from the sensors (M1-M5).

The step S120, the cramp detector 120 of the cramp evaluating device 100 inputs the physiological signals S1 to the evaluating model 125 for calculating the probability P1 of cramp to calf muscle of the runner.

In step S130, the controller 150 determines whether the probability P1 is larger than the threshold. If yes, the process proceeds to the step S140. If not, the process proceeds back to the step S110. In step S110, the cramp evaluating device 100 continues to calculate the probability P1 of cramp to calf muscle. In another embodiment, when the probability P1 is less than the threshold, the controller 150 may control the notice unit 130 to output the safe signal S3.

In step S140, the controller 150 controls the notice unit 130 to output the warning signal S2 for warning the runner of cramp being possible to occur.

In another embodiment, the steps S130 and S140 of the cramp evaluating method of the cramp evaluating device 100 for calf muscle may be omitted.

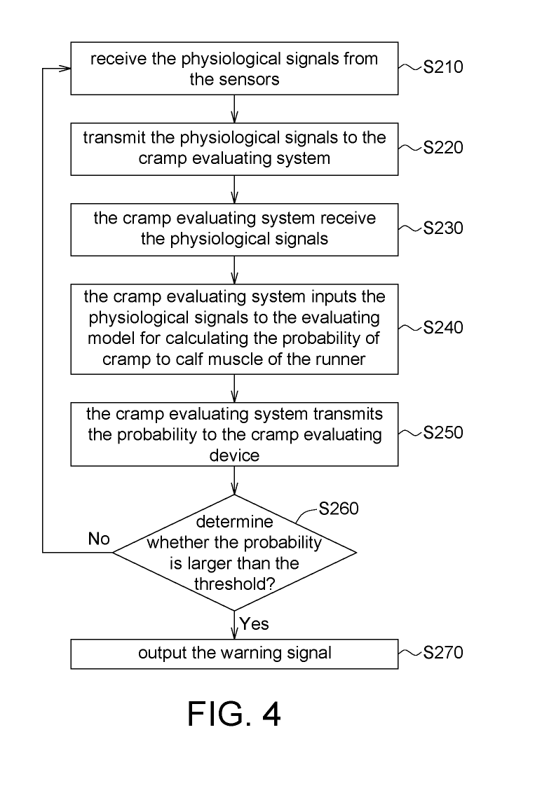

Referring to FIGS. 2 and 4, FIG. 4 shows a flowchart of a cramp evaluating method of the cramp evaluating device 200 for calf muscle of FIG. 2.

In step S210, the physiological signal receiver 110 of the cramp evaluating device 200 receives the physiological signals S1 from the sensors (M1-M5).

In step S220, the controller 150 of the cramp evaluating device 200 controls the device wireless transceiver 140 to transmit the physiological signals S1 to the cramp evaluating system 10.

In step S230, the system wireless transceiver 11 of the cramp evaluating system 10 receives the physiological signals S1.

In step S240, the cramp detector 120 of the cramp evaluating system 10 inputs the physiological signals S1 to the evaluating model 125 for calculating the probability P1 of cramp to calf muscle of the runner.

In step S250, the system wireless transceiver 11 of the cramp evaluating system 10 transmits the probability P1 to the cramp evaluating device 200.

In step S260, the device wireless transceiver 140 of the cramp evaluating device 200 receives the probability P1. Then, the controller 150 of the cramp evaluating device 200 determines whether the probability P1 is larger than the threshold. If yes, the process proceeds to the step S270. If not, the process proceeds back to the step S210. In step S210, the cramp evaluating device 200 continues to receive the physiological signals S1, and the cramp evaluating system 10 continues to calculate the probability P1 of cramp to calf muscle.

In step S270, the controller 150 of the cramp evaluating device 200 controls the notice unit 130 to output the warning signal S2 for warning the runner of cramp being possible to occur.

In another embodiment, the step S250 of FIG. 4 may be omitted, and it may be replaced by a step of the cramp evaluating system 10 determines whether the probability P1 is larger than the threshold. If yes, the cramp evaluating system 10 transmits the notice signal to the cramp evaluating device 200, and then the cramp evaluating device 200 outputs the warning signal S2. If not, the process proceeds back to the step S210.

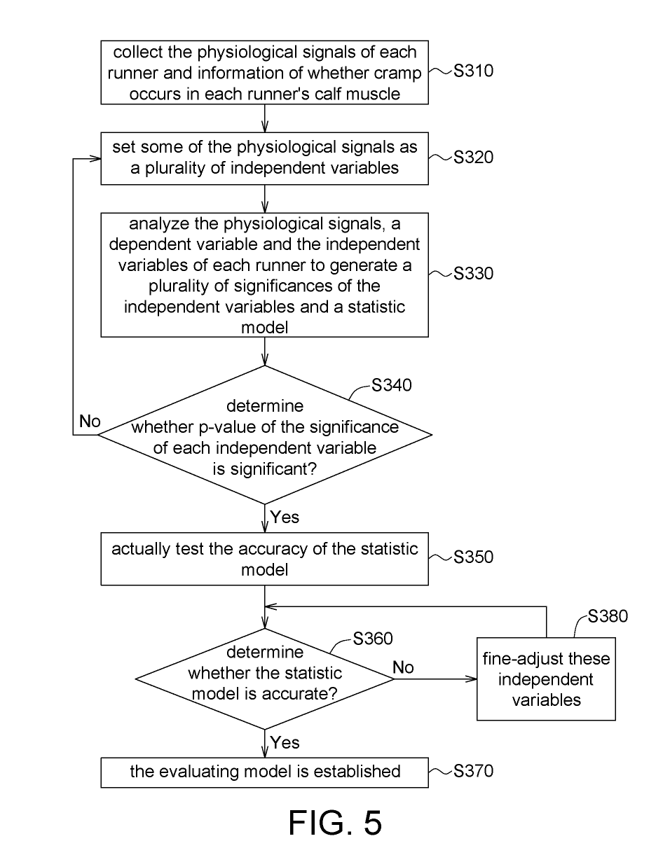

FIG. 5 shows a diagram of an establishing process of the evaluating model 125 according to an embodiment of the invention.

In step S310, the physiological signal receiver 110 of the cramp evaluating device 100 receives the physiological signals S1 from the sensors (M1-M5). The physiological signals S1 are, for example, at least two of mean power frequency, maximal volitional contraction, heart rate variability, root mean square, medium frequency, body temperature, foot pressure, Integrate EMG, critical velocity and AT/AeT.

In step S310, a learning machine (not shown) collects the physiological signals S1 of each runner and information of whether cramp occurs in each runner's calf muscle. These runners may be screened. For example, the number of the runners being 1,000 is taken for example, if the physiological signals S1 of 200 runners have no significance or meaning (such as the runner did not complete the whole process of testing, or signal interruption or divergence caused by runner's sensor offset or fall off), the physiological signals S1 of the 200 runners are excluded.

In step S320, some of the physiological signals S1 (for example, some of the types of the physiological signals S1) are set as a plurality of independent variables. The independent variables may be selected by the learning machine or man-made selection. For example, the mean power frequency, maximal volitional contraction and heart rate variability of the physiological signals S1 are set as the independent variables.

In step S330 the learning machine analyzes the physiological signals S1, a dependent variable and the independent variables of each runner to generate a plurality of significances of the independent variables and a statistic model using a machine learning algorithm, wherein the dependent variable means information of whether cramp occurs in each runner's calf muscle. If the cramp occurs in runner, the dependent variable is set as 1, for example. If the cramp does not occur in runner, the dependent variable is set as 0, for example. The dependent variable may be inputted by hand. In an embodiment, the machine learning algorithm is Logistic regression, for example.

In step S340, the learning machine determines whether p-value of the significance of each independent variable is significant. If yes, the process proceeds to the step S350. If not, the process proceeds back to the step S320. In step S320, others of the physiological signals S1 (for example, others of the types of the physiological signals S1) are set as the independent variables, wherein the set physiological signals S1 do not overlap completely or partly overlap with the previously selected physiological signals S1. For example, if p-value of the selected maximal volitional contraction is not significant, the maximal volitional contraction is replaced by another physiological signal S1 which is set as the significance of the independent variable. Alternatively, another group of the physiological signals S1 is set as the significance of the independent variables.

In step S350, the learning machine actually tests the accuracy of the statistic model. The test method is, for example, confusion matrix technology.

In step S360, the learning machine determines whether the statistic model is accurate. For example, the learning machine determines whether the statistic model is over fitting or under fitting. If the statistic model is over fitting and under fitting, it means that the statistic model is not accurate and then the process proceeds to step S380. If the statistic model is not over fitting and under fitting, it means that the statistic model is accurate and then the process proceeds to step S370. In step S370, the statistic model is set as the evaluating model 125.

In step S380, the learning machine fine-adjusts (or fine-tunes) these significances of the independent variables. For example, the learning machine, based on a significance of the independent variable, makes variable conversion for meeting or approximate meeting some of the conditions required for the analysis, such as normality conditions. The aforementioned variable conversion is, for example, way of a Box-Cox Transformation. In another embodiment, for the skewed data, the learning machine may take the form of logarithms, square root transformations, etc. for better distribution characteristics or other purposes. After step S380, the process proceeds back to step S360. In step S360, whether the statistic model is accurate is continued to be confirmed. Alternatively, the process may back to step S330 to re-select another physiological signal (another of the types of the physiological signals S1) as the independent variable.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A cramp evaluating device for calf muscle, adapted to evaluate a probability value of calf muscle of a runner wearing a number of sensors, and the cramp evaluating device comprising:
a physiological signal receiver configured to receive a plurality of physiological signals from the sensors; and
a cramp detector configured to input the physiological signals to an evaluation model to calculate the probability value of the calf muscle of the runner.

2. The cramp evaluating device according to claim 1, wherein the physiological signals include at least two of mean power frequency (MPF), maximal volitional contraction (MVC), heart rate variability (HRV), root mean square (RMS), medium frequency (MF), body temperature and foot pressure.

3. The cramp evaluating device according to claim 1, further comprising:
a notice unit configured to output a warning signal when the probability value of is larger than a threshold.

4. A cramp evaluating device for calf muscle, adapted to evaluate a probability value of calf muscle of a runner wearing a number of sensors, and the cramp evaluating device comprising:
a physiological signal receiver configured to receive a plurality of physiological signals from the sensors; and
a device wireless signal transceiver configured to transmit the physiological signals to a cramp evaluating system for calf muscle, wherein the cramp evaluating system inputs the physiological signals to an evaluation model for calculating the probability value of the calf muscle of the runner, and the device wireless signal transceiver is configured to receive the probability value.

5. The cramp evaluating device according to claim 4, wherein the physiological signals include at least two of mean power frequency, maximal volitional contraction, heart rate variability, root mean square, medium frequency, body temperature and foot pressure.

6. The cramp evaluating device according to claim 4, further comprising:
a notice unit configured to output a warning signal when the probability value of is larger than a threshold.

7. A cramp evaluating system for calf muscle, adapted to evaluate a probability value of calf muscle of a runner wearing a number of sensors, and the cramp evaluating system comprising:
a system wireless signal transceiver configured to receive a plurality of physiological signals from the sensors; and
a cramp detector configured to input the physiological signals to an evaluation model to calculate the probability value of the calf muscle of the runner;
wherein the system wireless signal transceiver is further configured to transmit the probability value to a cramp evaluating device.

8. A cramp evaluating method for calf muscle, adapted to evaluate a probability value of calf muscle of a runner wearing a number of sensors, and the cramp evaluating method comprising:
receiving a plurality of physiological signals from the sensors; and
inputting the physiological signals to an evaluation model to calculate the probability value of the calf muscle of the runner.

9. The cramp evaluating method according to claim 8, wherein the physiological signals include at least two of mean power frequency, maximal volitional contraction, heart rate variability, root mean square, medium frequency, body temperature and foot pressure.

10. The cramp evaluating method according to claim 8, further comprising an evaluation model establishing process, wherein the evaluation model establishing process comprises:
collecting the physiological signals of each runner and information of whether cramp occurs in each runner's calf muscle;
setting some of the physiological signals as a plurality of independent variables;
analyzing the physiological signals, a dependent variable and the independent variables of each runner to generate a plurality of significances of the independent variables and a statistic model using a machine learning algorithm, wherein the dependent variable means information of whether cramp occurs in each runner's calf muscle;
determining whether p-value of the significance of each independent variable is significant;
if all of the significances of the independent variables are significant, actually testing an accuracy of the statistic model; and
if the statistic model is accurate, setting the statistic model as the evaluation model.

11. The cramp evaluating method according to claim 10, wherein the machine learning algorithm is Logistic regression.

12. The cramp evaluating method according to claim 10, wherein the step of actually testing the accuracy of the statistic model is achieved by confusion matrix technique.

13. The cramp evaluating method according to claim 10, wherein the evaluation model establishing process further comprises:
If the p-value of the significance of at least one of the independent variables is not significant, returning to the step of setting some of the physiological signals as the independent variables, wherein others of the physiological signals are set as the independent variables.

14. The cramp evaluating method according to claim 10, wherein the evaluation model establishing process further comprises:
If the statistic model is not accurate, fine-adjusting the independent variables.
